(19) United States
(12) Patent Application Publication
(10) Pub. No.: US 2013/0197189 A1
(43) Pub. Date: Aug. 1, 2013

(54) Method For Synthesizing A Cyclic Multivalent Peptide Using A Thiol-Mediated Reaction

(71) Applicant: The Regents of the University of Colorado, A Body Corporate, Denver, CO (US)

(72) Inventors: Alex Aimetti, Superior, CO (US); Kristi Anseth, Boulder, CO (US); Cole DeForest, Boulder, CO (US)

(73) Assignee: The Regents of the University of Colorado, A Body Corporate, Denver, CO (US)

(21) Appl. No.: 13/711,293

(22) Filed: Dec. 11, 2012

Related U.S. Application Data
(63) Continuation of application No. PCT/US11/39938, filed on Jun. 10, 2011.
(60) Provisional application No. 61/354,070, filed on Jun. 11, 2010.

(51) Int. Cl. C07K 1/04

(52) U.S. Cl. CPC C07K 1/04
USPC 530/321

(57) ABSTRACT

A method has been developed for the formation of multivalent cyclic peptides. This procedure exploits on-resin peptide cyclization using a photoinitiated thiol-ene click reaction and subsequent clustering using thiol-yne photochemistry. Both reactions utilize the sulfhydrl group on natural cysteine amino acids to participate in the thiol-mediated reactions.

### RELATED APPLICATIONS

This application is a continuation of PCT Patent Application Serial No. PCT/US2011/039938, filed Jun. 10, 2011, which claims benefit of priority to U.S. Provisional Patent Application Ser. No. 61/354,070 filed Jun. 11, 2010, the contents of which are hereby incorporated by reference to the same extent as though fully replicated herein.

### GOVERNMENT INTERESTS

This invention was made with government support under grant number R01 DK076084 awarded by the National Institutes of Health. The government has certain rights in the invention.

### BACKGROUND

Peptides and their application as potential therapeutics have gained interest in the fields of chemical biology and drug discovery due to their ability to bind to highly selective targets that are of therapeutic interest (24). These biomolecules are capable of accessing both intra- and extracellular targets thereby expanding the therapeutic options for a given disease. However, peptides derived as fragments from whole proteins may not perform at or near the level of the native protein because the peptides may be unstable or lack the conformation of the larger proteins.

Researchers have made great advances in addressing the inherent disadvantages related to peptide therapeutics. Peptide macrocyclization, including peptide stapling, has rendered the biomolecules more potent in binding to their intended target (5,6). Additionally, peptides with a constrained conformation are more resistant to proteolytic degradation and are capable of achieving in vivo half-lives up to 24 hours (6). Macrocyclization has been reported, either on-resin or in solution, using a variety of ligation chemistries. The synthesis of cyclic peptides has been traditionally achieved by the formation of disulfide, amide, ester, olefin, and carbon-carbon bonds (27, 28, 29, 30, 31). These reactions can be performed on-resin to facilitate purification and to achieve the pseudodilution effect that promotes intramolecular ligation.

Additionally, multivalent interactions are known to play a critical role in many biological processes. The synthesis of multivalent peptides has further enhanced the interaction of individual ligands with their receptors. Multiple antigenic peptides (MAPs) were discovered by Tam et al. and utilized a branched lysine core (9). Although this approach is an elegant way to build multivalent peptides on the solid phase, the presence of single amino acid deletions makes purification difficult.

More recently, researchers have looked for highly efficient, chemoselective reactions to combine multiple peptides to a single core, or handle. “Click” reactions, defined by Sharpless, met the need for highly specific, rapid reactions between two functional groups in high yield for the synthesis of multivalent peptides. Recent reports exploit the copper (I)-catalyzed Huisgen azide-alkyne 1,3-dipolar cycloaddition reaction as an effective way to conjugate azido containing peptides to a core molecule with multiple alkyne handles (32). These peptide dendrimers exhibit enhanced potency in relation to their monomeric counterpart.

### SUMMARY

The presently disclosed instrumentalities overcome some of the problems outlined above and advance the art by providing a unique method for the formation of multivalent cyclic peptides. In one aspect, a peptide cyclization technique is disclosed by using a photoinitiated thiol-ene click reaction which may occur on resin. In another aspect, the thiol-ene click reaction may be followed by a thiol-yne photo reaction to create multivalent cyclic peptides. Both the thiol-ene and the thiol-yne reactions may utilize the sulfhydryl group on the cysteine amino acid residue(s) to participate in the thiol-mediated reactions. The radical-mediated reactions disclosed herein may be used for the formation of multivalent peptides, including oligopeptides up to entire proteins, as well as carbohydrates and other pharmaceutically active molecules. For instance, therapeutically active compounds such as peptides, carbohydrates or other pharmaceutically active molecules may be attached to the multivalent molecules using schemes similar to those described in U.S. patent application Ser. No. 11/956,719, which is hereby incorporated by reference into this disclosure.

In an embodiment, the disclosed process may include a step of forming an on-resin cyclic peptide using thiol-ene photo reaction, which may include a step of providing a linear peptide having at least one free thiol group and at least one free unsaturated carbon-carbon bond, which may be an alkene or alkyne group. In one aspect, the at least one free unsaturated carbon-carbon bond is an alkene group. The linear peptide may be attached to a solid support such as resin. The method may further include a step of forming a cyclic peptide in a photoreaction wherein the at least one free thiol group on the peptide reacts with the at least one alkene group.

In one aspect, the method may include a step of treating the linear peptide with a photoinitiator. After the photo reaction is finished, the cyclized peptide may be separated from the solid support. In one embodiment, the at least one free thiol group exists naturally in the cysteine residue on the natural sequence of the linear peptide. In another embodiment, the at least one free thiol group may exist in other amino acid residue, Aaa(SH), that has one or more free thiol group. One example of such Aaa(SH) is cysteine. The linear peptide may contain more than one such Aaa(SH) residues. Some or all Aaa(SH) residues may be protected before the thiol-ene reaction by capping with a protective group. Examples of such protective groups may include but are not limited to the monomethoxytrityl (Mmt) group. Certain Aaa(SH) may be deprotected immediately before the thiol-ene reaction is to take place. Removal of the protective group(s) may be accomplished by using various agents such as 2% TFA/CH₂Cl₂, among others. Alternatively, one or more Aaa(SH) residues (e.g., cysteines) may be introduced into the sequence of the linear peptide at one or more desirable loci.

In one embodiment, an Aaa(SH) residue that is designed to participate in the thiol-ene reaction may be capped with the Mmt protecting group and may be selectively deprotected on resin while other protecting groups remain intact. For example, other Aaa(SH) residues not designed to participate in the reaction may be protected with a trityl (Trt) protecting group that is labile under strong acid conditions.

In another embodiment, various agents may be used to introduce one or more alkene groups to the linear peptide. In one aspect, commercially available Fmoc-Lys(Alloc)-OH may be used as a building block to introduce an allyl ester within the peptide sequence. Alternatively, a strained, bicyclic alkene (norbornene) may be incorporated orthogonally to the peptide backbone.

The linear peptide may have the formula of

(Aaa)ₓ-Aaa(SH)-(Aaa)_{y}-Aaa(R)-(Aaa)_{z} (I)

or

(Aaa)ₓ-Aaa(R)-(Aaa)_{y}-Aaa(SH)-(Aaa)_{z} (II)

Aaa in (Aaa)ₓ, (Aaa)_{y}, (Aaa)_{z} or Aaa(R) may be any amino acid residue having an amine group and a carboxylic group, or isomers or derivatives thereof. Aaa may be an amino acid that occurs in proteins that exist in nature. Alternatively, Aaa may be an amino acid that does not occur in natural proteins. Aaa may also be an artificially synthesized amino acid. In one embodiment, the Aaa in Aaa(R) of Formula I or Formula II may be an amino acid with a side chain that contains an amino group. In another embodiment, the Aaa in Aaa(R) of Formula I or Formula II is a lysine residue. Aaa(SH) is an amino acid residue having at least one free thiol group. The R group may be a side chain containing an unsaturated carbon-carbon bond. In one embodiment, the R group may contain an alkene or alkyne. In another embodiment, the alkene may be attached to the Aaa residue forming an ester or an amide. In yet another embodiment, the alkene may be attached directly to the alpha-carbon of the Aaa residue in the Aaa(R) group of Formula I or Formula II. The alkene in the R group may be a linear alkene, a cyclic alkene or combination thereof. By way of example, the alkene in R may be norbornene or a linear alkene having the formula of CₙH₂ₙ, wherein n is an integer between 2 and 20, and more preferably, between 2 and 6.

(Aaa)ₓ, (Aaa)_{y} and (Aaa)_{z} may each be a string of amino acids with x, y and z indicating the length of each string, respectively. The subscripts x and z are each an integer between 0 and 100, and y may be an integer between 1 and 1,000, more preferably between 2 and 100. Preferably, x, y and z are all less than 50. When x is 0, no amino acid exists at the position of (Aaa)ₓ. Similarly, when z is 0, no amino acid exists at the position of (Aaa)_{z}. When x or z is 1, it means one single amino acid exists in the (Aaa)ₓ or (Aaa)_{z} position, and so on. Within each amino acid string of (Aaa)ₓ, (Aaa)_{y} or (Aaa)_{z}, the amino acids, namely, Aaa, may be the same or different. When a string is made up of different amino acids, these amino acids may form different sequences through permutation. (Aaa)ₓ, (Aaa)_{y} and (Aaa)_{z} may have the same or different sequences. In the thiol-ene photo reaction, after a photoinitiator is added and the reaction mixture is exposed to light, the thiol group on the Aaa(SH) reacts with the alkene on the R group thereby forming a cyclic peptide. In another aspect, the (Aaa)_{y} may contain one or more repeats of a small peptide containing 3 amino acids having the sequence of Arg-Gly-Asp (RGD). In another embodiment, the linear peptide is Ac-C(Mmt)RGDSfK(alkene). In another embodiment, the linear peptide is Ac-C(Mmt)RGDSfK(alkyne).

In another embodiment, a method of forming a cyclic, multivalent peptide using sequential thiol-ene/thiol-yne photo reactions is disclosed. This method may include the step of coupling one or more molecules having at least one alkyne to one or more peptide cores. The resulting one or more alkyne-functionalized-peptide cores may subsequently react with one or more Aaa(SH) (thiol) residues on the peptides to be multimerized. The method may further include a step of forming the multivalent cyclic peptide by coupling two or more peptides to the peptide core(s) having the alkyne group. In a preferred embodiment, the two or more peptides have at least one free thiol group and the coupling occurs by the free thiol group attacking the alkyne on the peptide core, The two or more peptides may be a linear peptide, a cyclic peptide or combination thereof. The two or more peptides may be the same or they may be different. The reaction may take place on a solid support such as a resin material, a polymer, or a hydrogel, or the reaction may take place in solution. The method may also include the steps of treating the reaction mixture with a photoinitiator and exposing the reaction mixture to light to form the multivalent cyclic peptide.

In another aspect, the method may further include the step of cleaving the multivalent cyclized peptide from the solid support to obtain the cyclic peptide product without the solid support.

In another embodiment of this disclosure, one or more cyclic peptides may be formed in an intra-molecule thiol-ene reaction where at least one thiol group attacks an unsaturated C—C bond on the same molecule that is attached on resin. The one or more cyclized peptides may be cleaved off the resin and be used in a subsequent thiol-yne reaction to form a multivalent cyclic peptide. Briefly, a multivalent cyclic peptide may be formed by coupling one or more peptides prepared in the thiol-ene reaction described above to one or more peptide cores. The one or more peptides preferably have at least one or more free thiol groups and the one or more peptide cores preferably have at least one alkyne. When the photoinitiator is added to the reaction mixture and the mixture is exposed to light of certain wavelength and intensity, the thiol-yne reaction may take place where one or more free thiol groups on the one or more peptides react with the at least one alkyne on the peptide core. The one or more peptides may be either linear or cyclic peptides. For instance, the one or more peptides may be formed through an internal thiol-ene reaction within a linear peptide of Formula I or Formula II.

In another embodiment, the solid support is polyethylene glycol (PEG) or a PEG-based hydrogel. PEG-based hydrogels represent a class of biomaterials having increasing applications in many fields, such as drug delivery and regenerative medicine. PEG is attracting more and more interest primarily due to its hydrophilic and inert properties. Peptides have been successfully incorporated within PEG hydrogels to serve as recognized biomolecules within a synthetic polymer platform.

In one aspect, the presently disclosed methodology may allow cyclic peptides to be synthesized on PEG. In another aspect, linear or cyclic peptides may be conjugated onto PEG after being synthesized. The peptides may also be engineered to respond to cellular stimuli or to have enhanced binding to biological molecules.

In one embodiment, the various components for forming a cyclic peptide or a multivalent cyclic peptide may be provided as a kit. For instance, the kit may contain a linear peptide, a solid support, a photoinitiator, a linker, among others.

The various peptides disclosed herein may be used alone or as one of the ingredients of a composition, along with other ingredients, solvents, carrier, excipients, etc.

### BRIEF DESCRIPTION OF THE DRAWINGS

FIG. 1 shows a synthetic route to form multivalent peptides using thiol-ene photochemistry.

FIG. 2A shows the characterization of Ac-CRGDSfK(Alloc)-NH2 linear(1) and cyclic(2) peptides by RP-HPLC. FIG. 2B shows Maldi-TOF mass spectral analyses of Ac-CRGDSfK(Alloc)-NH2 linear(1) and cyclic(2) peptides.

FIG. 3 shows an ¹H NMR of linear Ac-CRGDSfK(Alloc)-NH₂.

FIG. 4 shows an ¹H NMR of cyclic Ac-c[CRGDSfK(Alloc)]-NH_{2.}

FIG. 5 shows an HMBC spectrum of cyclic Ac-c[CRGDSfK(Alloc)]-NH_{2.}

FIG. 6 shows COSY/NOESY overlay of Ac-c[CRGDSfK(Alloc)]-NH_{2.}

FIG. 7 shows a chemical drawing of cyclic Ac-c[CRGDSfK(Alloc)[-NH_{2.}

FIG. 8 shows MALDI spectra for linear(1) and cyclic(2) Ac-CRGDSfK(Norbornene)-NH₂ respectively.

FIG. 9 shows the chemical structure of cyclic Ac-c[CRGDSfK(Norbornene)]-NH_{2.}

FIG. 10 shows ¹H NMR of cyclic Ac-c[CRGDSfK(Norbornene)]-NH_{2.}

FIG. 11 show HMBC spectrum of cyclic Ac-c[CRGDSfK(Norbornene)]-NH_{2.}

FIG. 12 shows gDQF-COSY spectrum of cyclic Ac-c[CRGDSfK(Noroborene)]-NH_{2.}

FIG. 13 shows a NOESY spectrum of cyclic Ac-c[CRGDSfK(Norbornene)]-NH_{2.}

FIGS. 14A, 14B and 14C show various NMR spectra of cyclic Ac-c[CRGDSfK(Norbornene)]-NH₂, and highlights the correlations within the molecule.

FIG. 15A shows the chemical structure of 5-norbornene-2-carboxylic acid. FIG. 15B shows NMR spectrum confirming bond correlations across the thioester bond. FIG. 15C shows Reverse Phase-HPLC chromatogram highlighting varying elution times between cyclic and linear peptides.

FIG. 16 shows the inhibition of fibrinogen binding to GPIIb/IIa in the presence of cyclic RGD derivatives formed via thiol-ene click chemistry.

FIG. 17 Shows the synthetic route to form multivalent peptides using thiol-yne photochemistry.

FIG. 18A Shows the evolution of compound #4 (linear RGD tetramer)determined by RP-HPLC. FIG. 18B shows that Peak C of FIG. 18A corresponds to a single peptide addition to a single alkyne as determined by ¹H NMR.

FIG. 19 shows the incorporation of cyclic and linear RGD within PEG hydrogels and its effect on encapsulated MIN6 cell metabolic activity at various concentrations and conformations.

FIG. 20 shows the incorporation of cyclic and linear RGD within PEG hydrogels and the clustered RGD effect on encapsulated MIN6 cell metabolic activity.

FIG. 21(a) shows a synthetic route to form cyclic RGD containing a free thiol. Conditions: (i) Thiolene photoreaction to form cyclic peptide. (ii) Deprotect Fmoc. (iii) Couple glycine, glycine, cysteine using standing coupling chemistries. (iv) Deprotect/cleave peptide from resin. FIG. 21(b) shows a synthetic route to form cyclic RGD dimer containing a free thiol.

### DETAILED DESCRIPTION

As used herein “peptide” refers to a chemical compound comprised of two or more amino acids covalently bonded together.

As used herein, the term “coupling” refers to forming a covalent bond between two molecules.

As used herein, an “unsaturated carbon-carbon bond(s)” is a chemical bond that contains carbon-carbon double bonds or triple bonds such as in alkenes or alkynes.

As used herein a “radical-mediated reaction” is a reaction in which an unpaired electron attacks an unsaturated carbon-carbon bond. Radical-mediated reactions may include photoinitiated (photoinitiation), thermal (thermal initiation) or redox initiated (redoz initiation) radical-mediated reactions.

As used herein “thiol” is a compound that contains the functional group composed of a sulfur-hydrogen bond. The general chemical structure of the thiol functional group is R—SH. Where “R” is a functional group, “S” is sulfur and “H” is hydrogen. The term “free thiol group” refers to a —SH group that is not bound by a protecting group, such as Mmt.

The term “α-amino acid” or “amino acid” refers to a molecule containing both an amino group and a carboxyl group bound to a carbon which is designated the α-carbon. Suitable amino acids include, without limitation, both the D- and L-isomers of the naturally-occurring amino acids, as well as non-naturally occurring amino acids prepared by organic synthesis or other metabolic routes. Unless the context specifically indicates otherwise, the term amino acid, as used herein, is intended to include amino acid analogs.

The term “naturally-occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

As used herein, a lower case amino acid letter abbreviation is used to depict the D steroisomer as opposed to the naturally occurring L form. Thus “F” refers to the L stereoisomer of the amino acid phenylalanine, while “f” refers to the D steroisomer of phenylalanine etc.

The term “amino acid analog” or “non-natural amino acid” refers to a molecule which is structurally similar to an amino acid and which can be substituted for an amino acid Amino acid analogs include, without limitation, compounds which are structurally identical to an amino acid, as defined herein, except for the inclusion of one or more additional methylene groups between the amino and carboxyl group (e.g., α-amino β-carboxy acids), or for the substitution of the amino or carboxy group by a similarly reactive group (e.g., substitution of the primary amine with a secondary or tertiary amine, or substitution or the carboxy group with an ester).

A “conservative amino acid substitution” is one in which the amino acid residue is replaced with an amino acid residue having a similar side chain Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H).

The term “amino acid side chain” refers to a moiety attached to the α-carbon in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, for example, those that do not occur in proteins in nature (e.g., an amino acid metabolite) or those that are made synthetically (e.g., an α, α di-substituted amino acid).

The term “peptide bond” refers to the amide [—C═O)—NH—] functionality with which individual amino acids are typically covalently bonded to each other in a peptide.

The phrases “macrocycle(s)”, “macrocyclic peptide(s),” and “cyclic peptide(s)” are used interchangeably herein to refer to both single cyclic and multi-cyclic compounds having one or more ring structures. The total number of atoms on each of such ring structures may be widely varied, e.g., in a range of from 3 to about 100 or more. Such single cyclic or multi-cyclic compound may further contain one or more linear functional groups, branched functional groups, and/or arched functional groups that bridge across a plane defined by a ring structure. In the case of multi-cyclic compounds having two or more ring structures, any pair of such ring structures may be separated from each another by a non-cyclic spacing structure, or the rings can be in side-by-side relationship to each another, sharing one chemical bond or one atom. The three-dimensional structures of such compounds can be characterized by any geometric shape, either regular or irregular, including, but not limited to, planar, cylindrical, semispherical, spherical, ovoidal, helical, pyriamidyl, etc. Specifically, such macrocyclic compounds may include cyclic peptides.

Peptide synthesis is the production of peptides, which are organic compounds in which multiple amino acids are linked via peptide bonds which are also known as amide bonds. The biological process of producing long peptides (proteins) is known as protein biosynthesis. Peptides are synthesized by coupling the carboxyl group or C-terminus of one amino acid to the amino group or N-terminus of another. Chemical peptide synthesis starts at the C-terminal end of the peptide and ends at the N-terminus This is the opposite of protein biosynthesis, which starts at the N-terminal end.

As used herein, a “peptide core” is a monomer, an oligomer or a polymer of amino acids. In an embodiment, the peptide core may be synthesized using any combination of amino acids . In another embodiment, the peptide core may contain from 1 to 10,000 amino acids, or from 1 to 5,000 amino acids, or from 1 to 1,000 amino acids, or more preferably, from 1 to 100 amino acids in length. In another aspect, the peptide core may contain a lysine residue or its derivatives. In another embodiment, the peptide core may contain one or more glycine residues.

Solid-phase peptide synthesis (SPPS) occurs on small solid beads which are composed of an insoluble porous matrix and generally treated with functional units (‘linkers’) on which peptide chains can be built.

One manner of making of the peptides described herein is SPPS. The C-terminal amino acid is attached to a cross-linked solid phase support (described below) such as a polystyrene resin via an acid labile bond with a linker molecule. This resin is insoluble in the solvents used for synthesis, making it relatively simple and fast to wash away excess reagents and by-products. The N-terminus is protected with a protecting group, which is stable in acid, but removable by base. Any side chain functional groups are protected with base stable, acid labile groups.

The term “solid support” or “solid phase” may be used to refer to a mechanically and chemically stable platform that may be utilized to conduct solid phase chemistry. By way of example, the solid support or solid phase may include resins, polymers, and gels such as hydrogels. The term “polymer” may be used to refer to a molecule made up of repeating units which may be obtained from either synthetic or natural sources. Suitable polymers may include biodegradable materials or non-biodegradable materials. Examples of the polymers may include but are not limited to polyethylene glycol (PEG), polyvinyl alcohol, poly(hydroxypropylmethacrylamide), polyacrylamide, polystyrene, chitosan, polyesters such as polycaprolactones, polyanhydrides, polyurethane, and poly(de-lactic coglycolic acid (PLGA), polyacrylic acid, haluronic acid, alginate, gelatin, dextran, poly(2-hydroxyethyl methacrylate), or carboxymethyl cellulose. Hydrogels may be formed by chemically or physically crosslinking any of the above mentioned polymers.

Suitable resins may include but are not limited to polystyrene resins, polyamide resins such as, but not limited to MBHA Rink Amide resin; PEG (polyethylene glycol) hybrid polystyrene resin such as but not limited to Tentagel resin; and PEG based resin, such as but not limited to ChemMatrix®.

The term “linker” when used in reference to solid phase chemistry refers to a chemical group that is bonded covalently to a solid support and is attached between the support and the substrate typically in order to permit the release (cleavage) of the substrate from the solid support. However, it can also be used to impart stability to the bond to the solid support or merely as a spacer element. Many solid supports are available commercially with linkers already attached.

As used herein, “alkynes” are hydrocarbons that have a triple bond between two carbon atoms, with the general formula CₙH₂ₙ₋₂.

As used herein, “alkenes” are hydrocarbons that have at least one carbon-to-carbon double bond, with the general formula CₙH₂ₙ.

Norbornene or norbornylene or norcamphene is a bridged cyclic hydrocarbon. The molecule consists of a cyclohexene ring bridged with a methylene group in the para position. The molecule carries a double bond (alkene) which induces significant ring strain and significant reactivity.

The term “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in “Protective Groups in Organic Synthesis,” Theodora W. Greene and Peter G. Wuts, editors, John Wiley & Sons, New York, 3^{rd} edition, 1999 [ISBN 0471160199]. Examples of protecting groups include, but are not limited to, phthalimido, trichloroacetyl, benzyloxycarbonyl, tert-butoxycarbonyl, and adamantyloxycarbonyl, allyloxycarbonyl (Alloc), benzyloxycarbonyl (Cbz), 9-fluorenylmethoxycarbonyl (Fmoc), tert-butoxycarbonyl (Boc), α,α-dimethyl-3,5-dimethoxybenzyloxycarbonyl (Ddz), acetyl, tert-butyldimethylsilyl (TBDMS), trityl (Trt), tert-butyl, tetrahydropyranyl (THP), methyl ester, tert-butyl ester, benzyl ester, trimethylsilylethyl ester, and 2,2,2-trichloroethyl ester.

Thiol-ene/Thiol-yne “click reactions” involve the addition of a S—H bond across a double or triple bond respectively by either a free radical or ionic mechanism.

As used herein a “photoinitiator” is any chemical compound that decomposes into free radicals or cations when exposed to light. Many types of photoinitiators are well known in the art. As disclosed herein a photoinitiator may be, but not limited to, 2,2-dimethoxy-2-phenylacetophenone (DMPA), diphenyl ketone, 2,4,6-Trimethylbenzoyl-diphenyl phosphine, acetophenone, benzyl, dibenzosuberenone, phenanthrenequinone, lithium phenyl-2,4,6-trimethylbenzoylphosphinate (LAP) and combinations thereof.

Photoinitiators are generally divided into two categories, Type I photoinitiators and Type II photoinitiators. Type I photoinitiators undergo a unimolecular bond cleavage upon irradiation to yield free radicals. Type II photoinitiators undergo a bimolecular reaction where the excited state of the photoinitiator interacts with a second molecule (a coinitiator) to generate free radicals.

The phrase “between . . . and” is inclusive. For instance, the phrase “between 5 and 8” refers to any value greater than or equal to 5 but less than or equal to 8. Likewise, the phrase “from . . . to”is inclusive. For instance, the phrase “from 5 to 8” refers to any value greater than or equal to 5 but less than or equal to 8.

As used herein, photoinitiators may be exposed to various wavelengths of light of various energies. In an embodiment, visible light may be used. In a preferred embodiment the light, may be Ultra Violet (UV). The wavelength of UV light may range from 10 nm to 400 nm, with energies ranging from 3 eV to 124 eV. In a more preferred embodiment the wavelength of UV light is 365 nm with an energy ranging between 15-20 mW cm⁻².

### EXAMPLE 1

### On-Resin Peptide Macrocyclization Using Thiol-Ene Click Chemistry

All reagents used were peptide synthesis grade unless otherwise noted. Fmoc-Cys(Mmt)-OH, Fmoc-Arg(Pbf)-OH, Fmoc-Gly-OH, Fmoc-Asp(OtBu)-OH, Fmoc-Ser(tBu)-OH, Fmoc-D-Phe-OH, Fmoc-Lys(Alloc)-OH, and Fmoc-Lys(Dde)-OH were obtained from Anaspec (San Jose, Calif.). Rink Amide MBHA resin (˜0.5 mmol/g resin) was purchased from Novabiochem (La Jolla, Calif.). Piperidine, 1,8-diazabicycloundec-7-ene (DBU), and 2,6-lutidine were obtained from Sigma-Aldrich (St. Louis, Mo.). N-methylmorpholine (NMM) and acetic anhydride were purchased from Fisher Scientific (Pittsburgh, Pa.). N-methylpyrolidone (NMP), dimethylformamide (DMF), and diisopropylethylamine (DIPEA) were purchased from Applied Biosystems (Foster City, Calif.). O-Benzotriazole-N,N,N′,N′-tetramethyl-uronium-hexafluoro-phosphate (HBTU) and 2-(1H-7-Azabenzotriazol-1-yl)-1,1,3,3-tetramethyl uronium hexafluorophosphate methanaminium (HATU) were obtained from Anaspec and ChemPep, Inc. (Wellington, Fla.), respectively. Hydrazine monohydrate, trifluoroacetic acid (TFA), triisopropylsilane (TIPS), phenol and 5-norbornene-2-carboxylic acid were purchased from Sigma-Aldrich. 2,2-dimethoxy-2-phenylacetophenone (DMPA) photoinitiator was obtained from Ciba (Tarrytown, N.Y.). Azobisisobutyronitrile (AIBN) was obtained from Sigma-Aldrich and used as a thermal initiator.

### General Peptide Synthesis

Peptides were built using Fmoc protected amino acids on a solid phase Rink Amide MBHA resin with an automated Tribute Peptide Synthesizer (Protein Technologies, Tucson, Ariz.). Peptides were synthesized on a 0.50 mmol scale. Fmoc deprotection occurred in 20% piperidine (v/v), 2% DBU (v/v) in NMP (2×5 min) 4 eq. (relative to resin) of activated amino acids (amino acid:HBTU:NMM; 1:1:2) were added to the Fmoc-deprotected resin and allowed to react for 35 minutes. After each coupling step, any unreacted sites were capped using 5% (v/v) acetic anhydride, 6% (v/v) 2,6-lutidine in NMP (10 min) After on-resin cyclization, peptides were cleaved from their solid support using TFA/TIPS/H₂O (95:2.5:2.5 (v/v)) and phenol (50 mg mL⁻¹ cleavage solution) and allowed to react for 2.5 hours. The filtrate was precipitated in and washed (3×) with chilled diethyl ether. The product was collected by centrifugation and allowed to dry in a desiccator for 2 hours. Peptides were purified using RP-HPLC (Waters Delta Prep 4000) with a C₁₈ prep column (Sunfire 30 mm×150 mm) using a 70-min linear (5-95%) gradient of acetonitrile in 0.1% trifluoroacetic acid. Peptides were characterized using analytical scale RP-HPLC (XBridge 4.6 mm×50 mm), MALDI-TOF MS (Applied Biosystem DE Voyager), and various NMR techniques.

### Ac-CRGDSfK(Alloc)-NH₂ Cyclization Using Thiol-Ene Chemistry

Following standard solid phase peptide synthesis, the cysteine residue was deprotected by cleaving the monomethoxytrityl (Mmt) group as described in Barlos, et al., Int. J. Pept. Protein Res. 1996, 47, 148-153. The availability of the free thiol was confirmed using a modified on-resin Ellman's assay. See Badyal, et al., Tetrahedron Lett. 2001, 42, 8531-8533. At this point, the resin was split into 3 equal parts. ⅓ of the resin was treated with TFA to cleave the peptide from the solid support, as described previously, to form the linear peptide (1). After purification and lyophilization, the linear peptide was solubilized in methanol (2 mM) with 1 eq. DMPA. The solution was irradiated with UV light (Omnicure) (365 nm, ˜20 mW cm⁻², 1 hour). DMPA was supplemented every 10 minutes to account for initiator consumption. The product was concentrated by rotary evaporation and subsequently precipitated in cold diethyl ether and washed (3×). Another portion of resin was transferred to a round bottom flask with a stir bar, purged with Ar, and allowed to swell in DMF for 30 minutes. DMPA photoinitiator (1 eq. to resin functional groups) was added to the round bottom flask and allowed to diffuse throughout the macroporous beads for 10 minutes. The resin was then exposed to UV light (365 nm, ˜20 mW cm⁻², 1 hour). DMPA was supplemented every 10 minutes to account for initiator consumption. The reaction was assumed complete when the Ellman's assay resulted in a negative test for free thiols. The resin was then washed with DCM (3×) and dried. The peptide was cleaved from the solid support as described above. The final portion of resin was added to a round bottom flask with 5 eq of AIBN in DMF. The reaction was performed at 65° C. for 48 hrs under Ar. The on-resin Ellman's assay was used to determine when the reaction was complete. The peptide was cleaved from the resin as described previously.

Characterization of Ac-CRGDSfK(Alloc)-NH₂ (Cyclic (2) and Linear (1)) Peptide

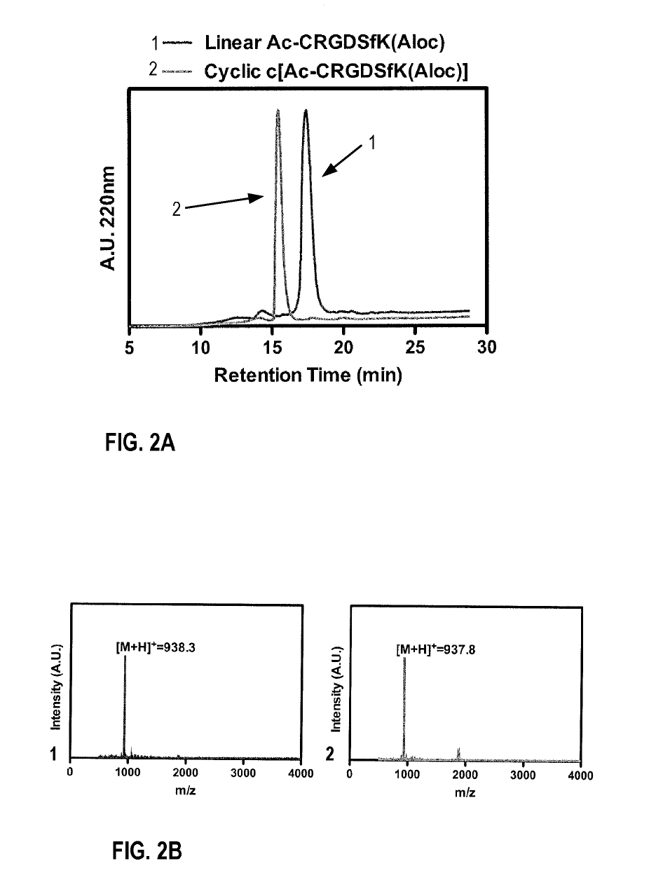

FIG. 2A shows an RP-HPLC chromatogram of linear (1) and cyclic (2) Ac-CRGDSfK(Alloc)-NH₂.

FIG. 2B shows Maldi-TOF spectra corresponding to linear (1) Ac-CRGDSfK(Alloc)-NH₂ and cyclic (2) Ac-c[CRGDSfK(Alloc)]-NH₂.

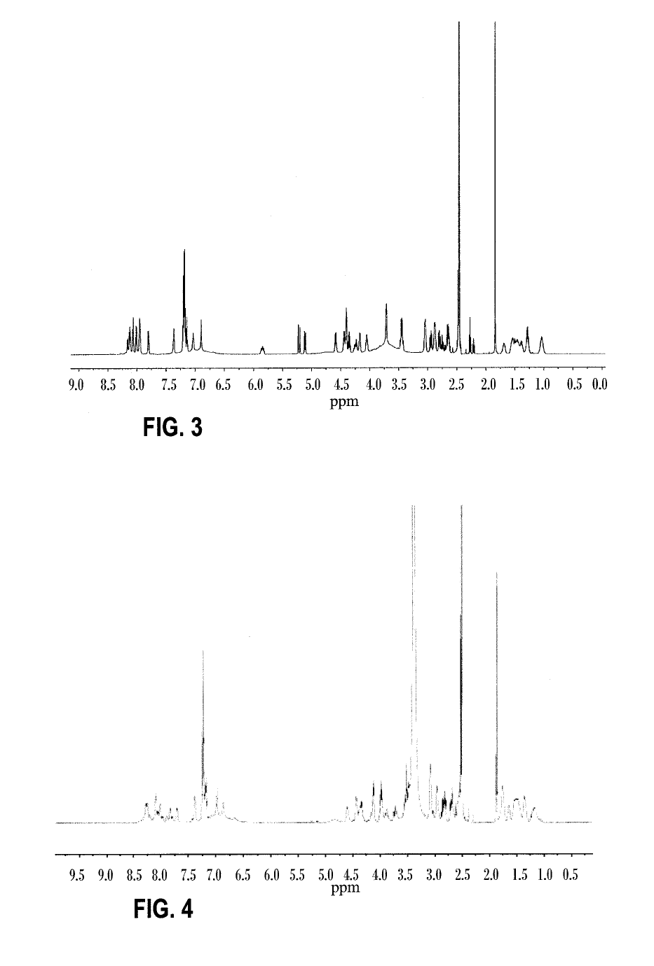

NMR spectra for Ac-CRGDSfK(Alloc)-NH₂ (cyclic (2) & linear (1)) peptides, as shown in FIG. 3 and FIG. 4, were obtained on a Varian Inova-600 NMR spectrometer operating at 599.71 MHz for ¹H observation. The instrument is equipped with a cryogenically cooled inverse geometry HCN ColdProbe, with the sample temperature maintained at 37.0° C. 2D-NMR experiments were performed as follows: COSY: gCOSY experiment, presented in magnitude mode; NOESY: gradient-enhanced, NOESY pulse sequence, with zeroquantum filter to minimize COSY cross peaks; HSQC: adiabatic gradient-HSQC using a matched adiabatic sweep to minimize loss of signal due to mismatch of J_{CH} coupling constant. ¹³C adiabatic decoupling utilizing a “clean-WURST” decoupling sequence was used; HMBC: a gradient-selected HMBC sequence was used, with data presented in magnitude mode, with the long-range C—H coupling delay optimized for a coupling constant of 8.5 Hz. All NMR spectra shown were processed and presented using the MestreNova 6.0 software package by Mestrelab Research, S.L. The NMR samples consisted of 3 mg of compound dissolved in DMSO-d6 (600 μL).

FIG. 4 further illustrates the disappearance of the vinyl protons (δ5.84-5.05) indicating a successful thiol-ene reaction.

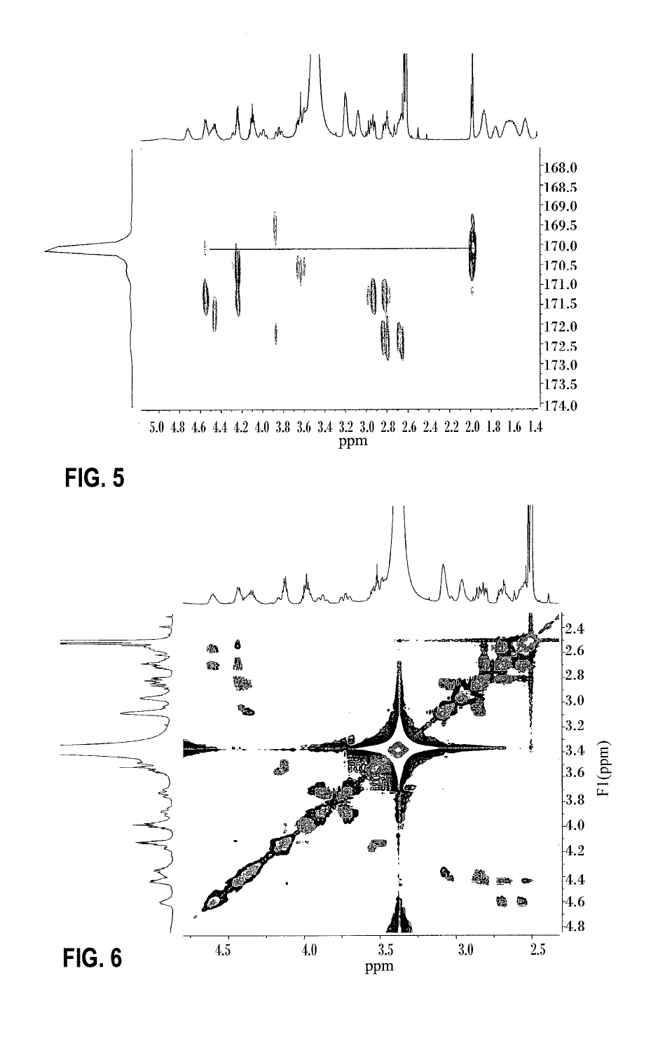

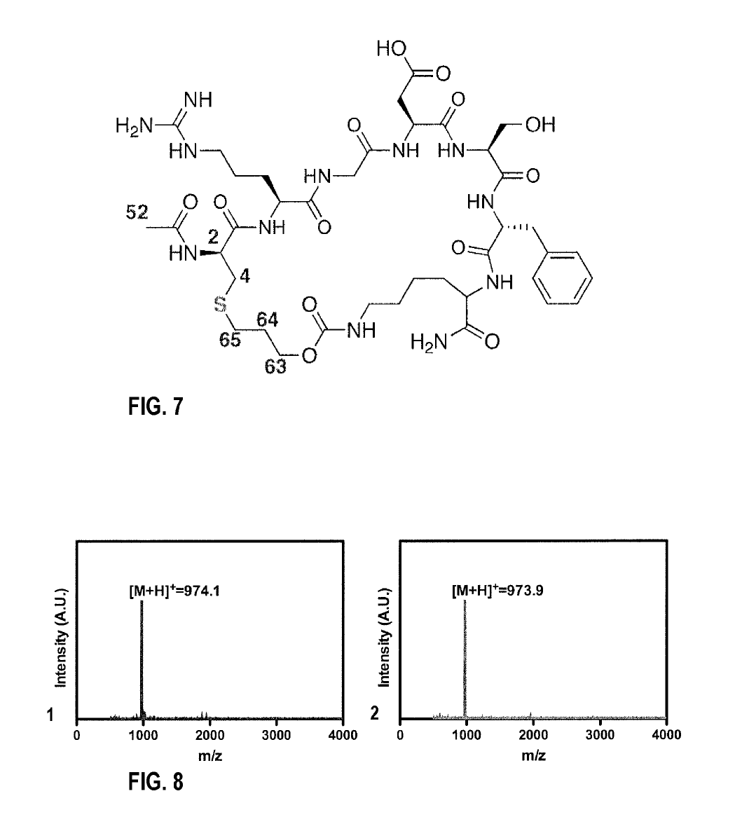

FIG. 5 shows the chemical shift assignments of the methyl protons on the acetyl group (“Ac”) (δ1.86, labeled “52” in FIG. 7) and the proton (δ4.56, labeled “2” in FIG. 7) bonded to the a-carbon of the cysteine residue correlated through the carbonyl of the acetyl group (170.1 ppm). Chemical shifts were assigned for protons (as illustrated in FIG. 7) proton 4 (δ2.84-2.69), proton 63 (δ3.98), proton 64 (δ1.76) and proton 65 (δ2.54) using a COSY spectrum.

A COSY/NOSY spectrum overlay, depicted in FIG. 6, illustrates NOE's that exist on protons adjacent to the thioether bond (proton 2→proton 65; proton 4→proton 65.

Ac-CRGDSfK(Norbornene)-NH₂ cyclization Using Thiol-Ene Chemistry

Ac-C(Mmt)RGDSfK(Dde)-resin was built on the solid phase as described previously. The Dde group was selectively deprotected on-resin (2% hydrazine in DMF), as described in Bycroft, et al., Journal of the Chemical Society-Chemical Communications 1993, 778-779. A positive Kaiser test confirmed the availability of the ε-amino group. 5-norbornene-2-carboxylic acid (4 eq to resin, 244 μL), HATU (4 eq to resin, 760 mg), and DIPEA (8 eq to resin, 696 μL) in DMF were added to the resin and allowed to react for 1 hour. A negative Kaiser test confirmed complete coupling. Next, the resin was washed (5×) with DCM and the Mmt protecting group was subsequently removed as described previously. The resin was partitioned similarly to the RGD derivatives containing the allyl ester. The on-resin and solution cyclization photoreaction was performed as described above with the exception that the thiol-ene photoreaction with the norbornene reached completion in 20 minutes. Thermal reactions were performed as described above. Peptides were cleaved from the resin as described previously.

FIG. 8 shows MALDI spectra for linear(1) and cyclic(2) Ac-CRGDSfK(Norbornene)-NH₂ respectively.

NMR spectra for linear (1) and cyclic (2) Ac-CRGDSfK(Norbornene)-NH₂ were obtained on a Varian VNMRS-800 NMR spectrometer operating at 799.33 MHz for ¹H observation. The instrument is equipped with a cryogenically cooled inverse geometry, HCN ColdProbe, with the sample temperature maintained at 25.0° C. This probe is optimized for enhanced ¹³C sensitivity via cryogenically cooled detection coil and receiver pre-amplifier, which yields an increase of approximately 6× in sensitivity for direct ¹³C observation, making it possible to observe ¹³C directly for this sample. 2D-NMR experiments were performed as follows: COSY: phase-sensitive DQF-COSY experiment, presented in pure-phase mode for improved resolution; NOESY: gradient-enhanced, NOESY pulse sequence, with zero-quantum filter to minimize COSY cross peaks; HSQC: adiabatic gradient-HSQC using a matched adiabatic sweep to minimize loss of signal due to mismatch of JCH coupling constant. ¹³C adiabatic decoupling using a “clean-WURST” decoupling sequence was used; HMBC: a gradient-selected HMBC sequence was used, with data presented in magnitude mode, with the long-range C—H coupling delay optimized for a coupling constant of 8.5 Hz. All NMR spectra shown were processed and presented using the MestreNova 6.0 software package by Mestrelab Research, S.L. The NMR samples consisted of 3 mg of compound dissolved in D₂O.

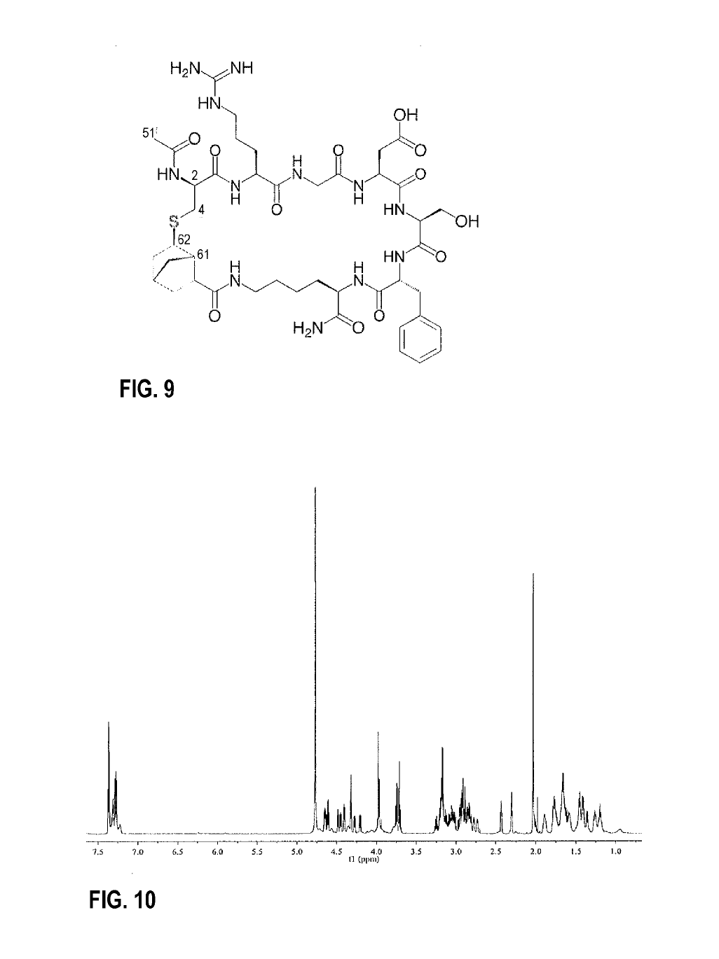

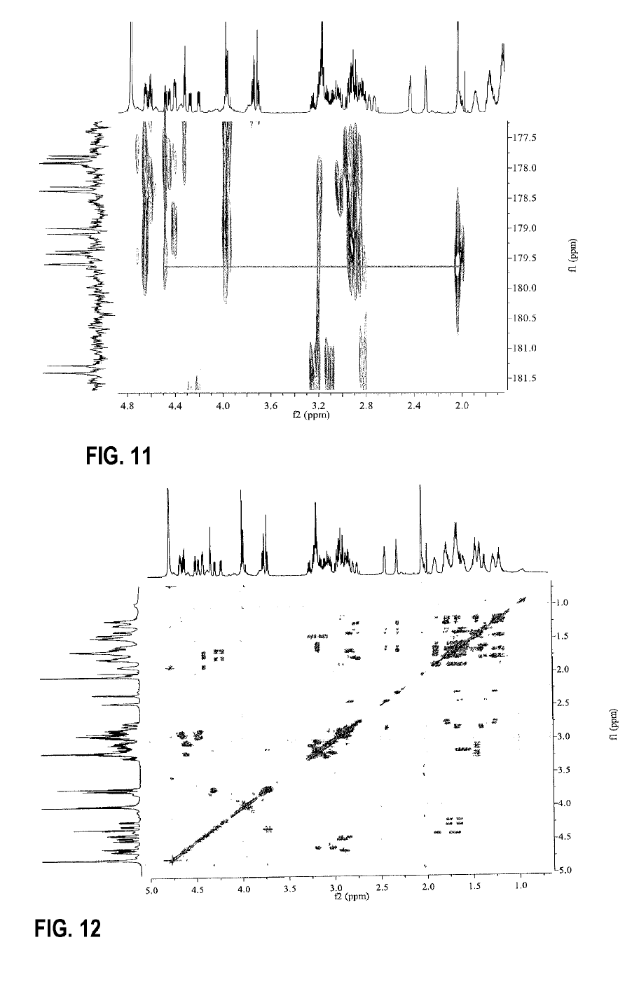

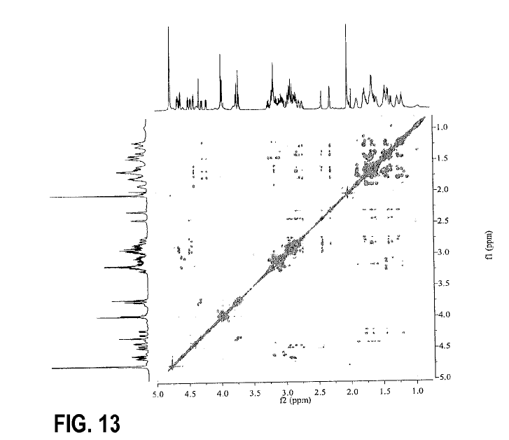

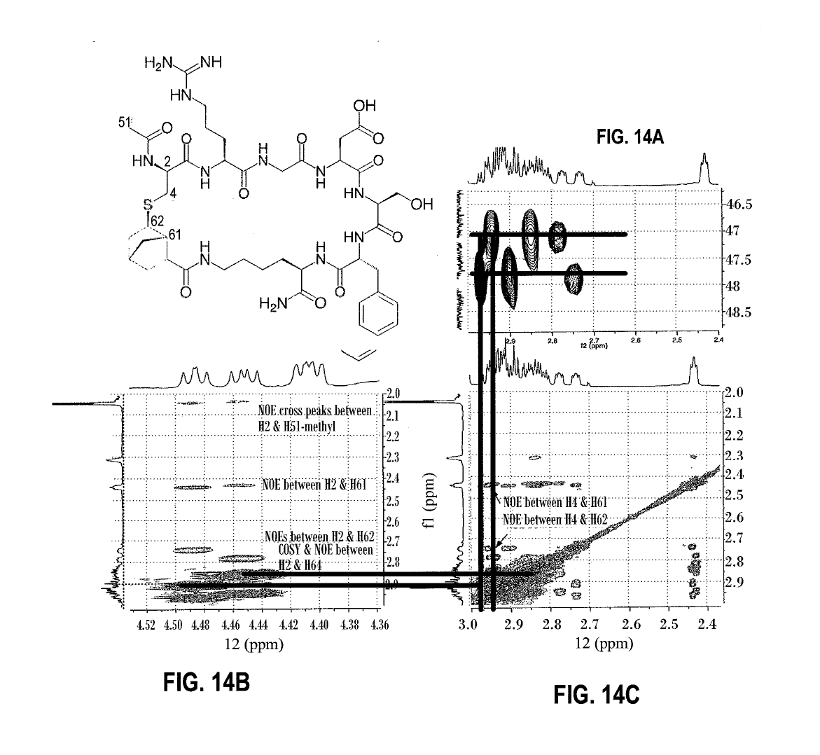

¹³C chemical shifts were referenced indirectly to Oppm in the ¹H NMR spectrum according to the IUPAC method as described in Harris, et al., Pure Appl. Chem. 2008, 80, 59-84.The ¹H NMR spectra were referenced using the residual HOD (water) signal at 4.77 ppm (HOD at 25° C.). The structure with numbered atoms is illustrated in FIG. 9. FIG. 11 is a HMBC spectrum illustrating the chemical shift assignment for proton 2 (δ1.86 ppm) correlated through the carbonyl on the acetyl group, which is assigned via the strong correlation to the acetyl-methyl protons. FIG. 12 is a gDQF-COSY spectrum used to assign protons 4 (diastereotopic; δ2.97-2.85 ppm) and 61 (δ2.44 ppm). FIG. 13 is a NOESY spectrum of cyclic Ac-c[CRGDSfK(Noroborene)]-NH₂. FIGS. 14A, 14B and 14C show various NMR spectra and highlights the correlations within the molecule (bold black lines). FIG. 14A is a HMBC/HSQC overlay showing the correlation through bonds between H4 and C62 (adjacent to thioether bond). FIGS. 14B and 14C are COSY/NOESY overlays showing the NOE's present between H2 and various protons (62, 61, and 51) and NOE's present between H4 and various protons (62 and 61).

### Fibrinogen Binding ELISA

A 96-well Maxisorp plate (Nunc) was coated with GPIIb/IIIa (10 μg mL⁻¹, 100 μL) and incubated at 4° C. overnight. The plate was washed (5×) with buffer (50 mM Tris-HCl, 100 mM NaCl, 2 mM CaCl₂, 0.05% Tween20, pH 7.4). Blocking solution (3.5% BSA, 100 μL) was added to the well and incubated at 37° C. for 3 hours. The plate was washed (5×) with buffer. Peptides (50 μL) and fibrinogen (40nM, 50 μL) were added to the plate and incubated at room temperature for 3 hours. The plate was washed (5×) and antibody (goat polyclonal to fibrinogen (HRP), 1:20,000 dilution, 100 μL) was added to the plate and incubated for 1 hour at room temperature. The plate was washed (5×) and substrate (2,2′-azino-bis(3-ethylbenzthiazoline-6-sulphonic acid, ABTS, 100 μL) was added to the wells and allowed to react for 5 minutes. The absorbance was measured (Perkin Elmer Wallac Victor2 1420 Multilabel Counter) at 405 nm. The data was analyzed using Graphpad Prism 5.

To generate a cyclic peptide formation on-resin, the natural amino acid, cysteine, was used as the thiol source for the reaction. However, peptides can be designed to contain cysteine residues that do not participate in the cyclization reaction by exploiting highly selective orthogonal Cys protecting groups. Various alkenes and their effect on the cyclization were studied. Commercially available Fmoc-Lys(Alloc)-OH was used as a building block to incorporate an allyl ester within the peptide sequence. The allyloxycarbonyl (Alloc) functional group is traditionally used as an orthogonal protecting group for lysine amino acids during peptide synthesis. However, recently the Lys(Alloc) monomer has been included within a peptide sequence to participate in a thiol-ene photoreaction to pattern within a hydrogel. This commercially available building block has been exploited as a facile method to incorporate an alkene within the peptide sequence. Alternatively, a strained, bicyclic alkene (norbornene) was incorporated orthogonally to the peptide backbone, as this alkene has a much higher reactivity. Arg-Gly-Asp (RGD), a peptide ligand of the αvβ3 integrin, was synthesized as a model peptide to demonstrate proof of concept.

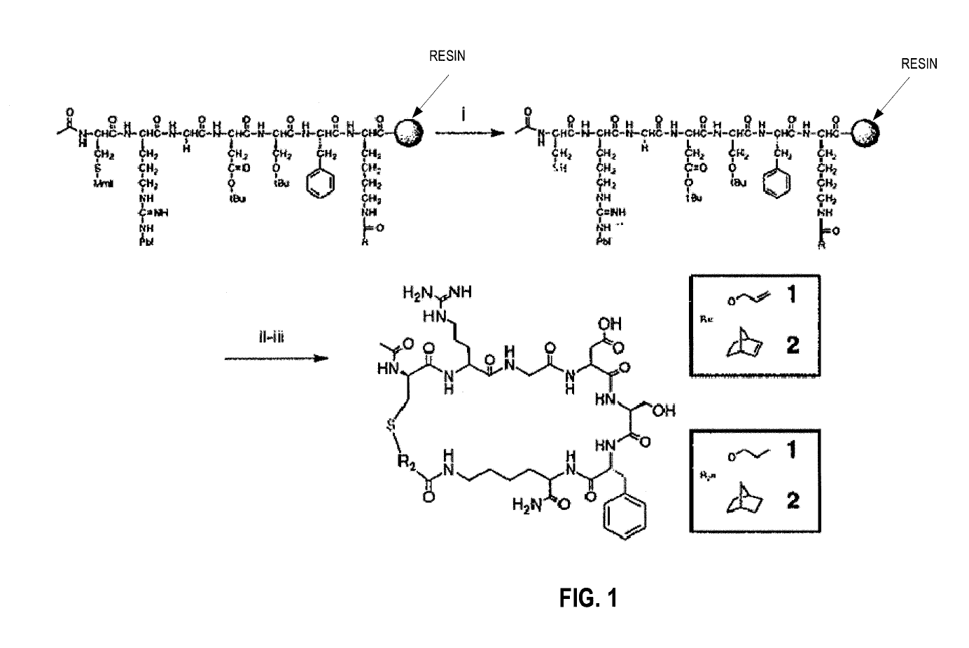

FIG. 1 illustrates the general scheme for cyclic peptide formation using an alkene target. Linear Ac-C(Mmt)RGDSfK(alkene) was built on the solid phase using Fmoc chemistry. The monomethoxytrityl (Mmt) sulfhydryl protecting group was selectively removed on resin using 2% TFA/CH₂Cl₂. A type I photoinitiator, 2,2-dimethoxy-2-phenylacetophenone (DMPA), was added to the peptidyl resin and exposed to 365 nm light. The reaction was well mixed to minimize the impact of light attenuation. An on-resin Ellman's test was utilized to qualitatively monitor the extent of thiol conversion. The reaction with the allyl ester reached completion (based on Ellman's test) at 1 hr. However, the reaction with the strained norbornene reached completion after just 20 min Cyclic Ac-c[CRGDSfK(Norbornene)]-NH₂) was recovered at 37% yield (calculated as the amount recovered after purification relative to crude mixture).

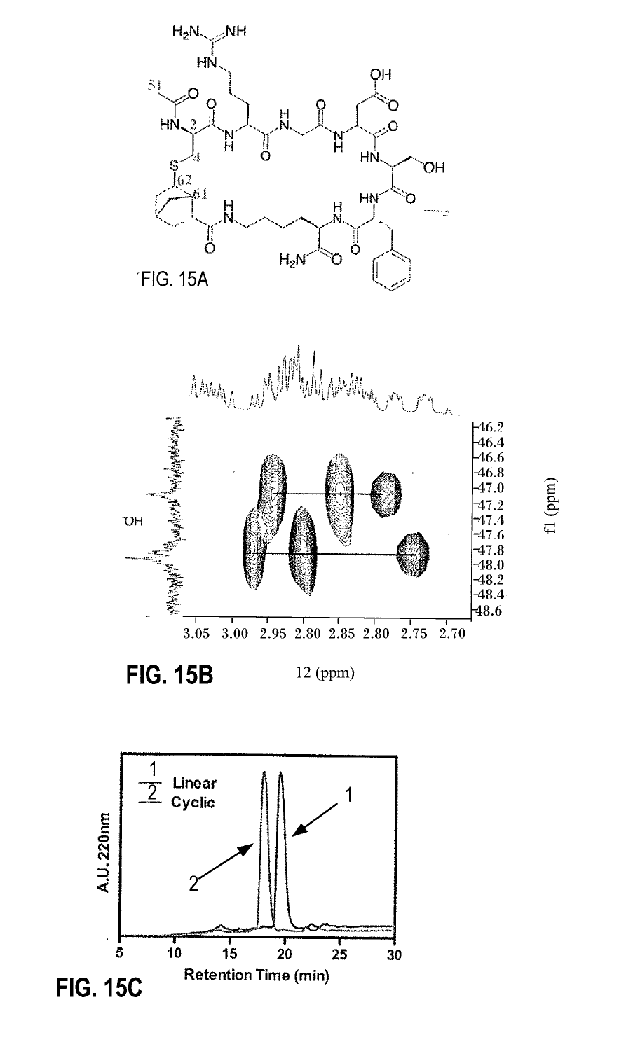

Cyclized products were characterized using various NMR techniques as disclosed herein. FIG. 15B illustrates unambiguous evidence of cyclization. The HMBC spectrum shows proton shifts at the #4 position (¹H δ2.97-2.85 ppm, diastereotopic protons influenced by the endo/exo conformation of the norbornane ring) that correlate with the carbon assignment at position #62 (¹³C δ47.87, 47.08 ppm) as shown using HSQC data. Further, FIG. 14B confirms the presence of NOEs between protons 4, 2, 51 and protons associated with the norbornane ring. As expected, cyclic peptides eluted earlier than its linear counterpart using RPHPLC (FIG. 15C). Spectral characterization for linear Ac-CRGDSfK(Alloc)-NH₂ and cyclic Ac-CRGDSfK(Alloc)-NH₂ is described above.

The IUPAC designations for cyclic Ac-CRGDSfK(Alloc)-NH₂ and Cyclic Ac-c[CRGDSfK(Norbornene)]-NH₂ are C₄₀H₆₁N₁₁O₁₃S and C₄₃H₆₄N₁₂O₁₂S, respectively.

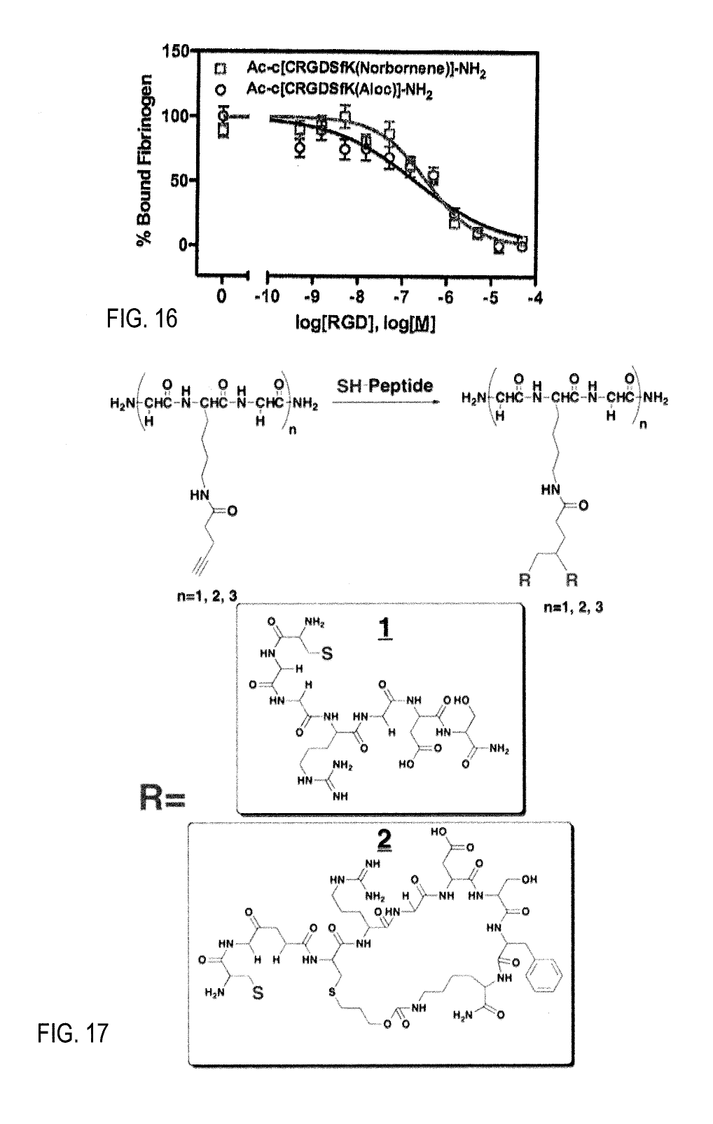

To confirm that the thiol-ene reaction did not exhibit deleterious effects on the cyclic peptides activity, a competitive binding ELISA was performed. Glycoprotein Iib-IIIa (GPIIb/IIIa) is present on platelets and its binding to fibrinogen has been associated with platelet aggregation. RGD has been shown to inhibit the binding of GPIIb/IIIa to fibrinogen. FIG. 16 shows the inhibition of fibrinogen binding to GPIIb/IIIa in response to cyclic peptide Ac-CRGDSfK(Alloc)-NH₂ and cyclic peptides Ac-c[CRGDSfK(Norbornene)]-NH₂ exhibiting an IC₅₀ values of 0.20±0.09 and 0.36±0.09 μM, respectively. The linear peptides were less potent in inhibiting fibrinogen binding (IC₅₀=1.41±0.28 μM).

The presently disclosed instrumentalities provide a unique method to exploit the thiol group of natural cysteine amino acids and allows for various alkenes to be incorporated orthogonal to the peptide backbone. Cyclic peptides were formed rapidly, from between around 20 minutes to around 1 hour depending on alkene functionality and were recovered in high yields (˜37%) relative to other on-resin techniques. In addition, the cyclic product formed via thiol-ene click chemistry retained its therapeutic efficacy.

### EXAMPLE 2

### Synthesis of Cyclic, Multivalent Arg-Gly-Asp Peptides Using Sequential Thiol-Ene/thiol-Yne Photoreaction

### Materials:

All reagents used were peptide synthesis grade unless otherwise noted. Fmoc-Cys(Mmt)-OH, Fmoc-Cys(Trt)-OH, Fmoc-Arg(Pbf)-OH, Fmoc-Gly-OH, Fmoc-Asp(OtBu)-OH, Fmoc-Ser(tBu)-OH, Fmoc-D-Phe-OH, Fmoc-Lys(Mtt)-OH, Fmoc-Lys(Alloc)-OH were obtained from Anaspec (San Jose, Calif.). Fmoc-Ahx-OH and Rink Amide MBHA resin (0.56 mmol/g resin) were purchased from Novabiochem (La Jolla, Calif.). Piperidine, 1,8-diazabicycloundec-7-ene (DBU), and 2,6-lutidine were obtained from Sigma-Aldrich (St. Louis, Mo.). N-methylmorpholine (NMM) and acetic anhydride were purchased from Fisher Scientific (Pittsburgh, Pa.). N-methylpyrolidone (NMP), dimethylformamide (DMF), and diisopropylethylamine (DIPEA) were purchased from Applied Biosystems (Foster City, Calif.). O-Benzotriazole-N,N,N′,N′-tetramethyluronium-hexafluoro-phosphate (HBTU) and 2-(1H-7-Azabenzotriazol-1-yl)-1,1,3,3-tetramethyl uronium hexafluorophosphate methanaminium (HATU) were obtained from Anaspec and ChemPep, Inc. (Wellington, Fla.), respectively. Trifluoroacetic acid (TFA), triisopropylsilane (TIPS), phenol, 4-pentynoic acid, and (human) fibrinogen were purchased from Sigma-Aldrich. 2,2-dimethoxy-2-phenylacetophenone (DMPA) photoinitiator was obtained from Ciba (Tarrytown, N.Y.). GPIIb/IIIa was obtained from Enzyme Research Laboratories (South Bend, Ind.). Fibrinogen antibody (HRP) was purchased from Abcam (Cambridge, Mass.).

### General Peptide Synthesis and Purification:

Peptides were built on the solid phase using an automated Tribute Peptide Synthesizer (Protein Technologies, Tucson, Ariz.). Peptides were synthesized on the 0.25-0.5 mmol scale. Fmoc deprotection was achieved using 20% piperidine in NMP (5 min×2). 4 eq. of Fmoc-protected amino acids were activated using HBTU/NMM (1:2, molar ratios of HBTU and NMM, respectively, in relation to amino acid) and added to the resin and allowed to react for 35 min. Any unreacted sites were acetylated using 5% (v/v) acetic anhydride/6% (v/v) 2,6-lutidine in NMP (10 min) Peptides were cleaved from their solid support using TFA/TIPS/H₂O (95:2.5:2.5 (v/v)) and phenol (50 mg mL⁻¹ cleavage solution) and allowed to react for 2 h. Peptides were precipitated in chilled diethyl ether and washed (3×). Product was allowed to dry in a desiccator for 2 h prior to HPLC purification. Peptides were purified using RP-HPLC (Waters Delta Prep 4000) with a C¹⁸ prep column (Sunfire 30 mm×150 mm) using a 70-min linear (5-95%) gradient of acetonitrile in 0.1% trifluoroacetic acid. Peptides were characterized using analytical scale RP-HPLC (XBridge 4.6 mm×50 mm), MALDI-TOF MS (Applied Biosystem DE Voyager), and ¹H NMR.

### Synthesis of H-GK(4-pa)G-NH2 (Core 1):

H-GK(Mtt)G-resin was synthesized as described in the General Peptide Synthesis section above. The Mtt group was selectively deprotected on resin as described previously. Briefly, 1.5% TFA in dichloromethane was added to the resin (30 sec.×9). A positive Kaiser test confirmed the availability of the free amine 4-pentynoic acid (4-pa; 5 eq. to resin) was then coupled to the ε-amino group using HATU/DIPEA in DMF and allowed to react for 2 hours under Ar. A negative Kaiser test confirmed successful coupling. Product was characterized using MALDI-TOF and ¹H NMR. MALDI: Calculated [M+H]+=340.4g/mol, found [M+H]+=340.7 g/mol. δ ¹H NMR (500 MHz, D₂O) 4.29 (1H, t, J 6.8), 3.92-3.83 (2H, m, J 17.2), 3.84 (2H, s), 3.17 (2H, t, J 6.3), 2.45 (2H, d, J 6.2), 2.39 (2H, t, J 6.7), 2.32 (1H, s), 1.85-1.66 (2H, m), 1.56-1.46 (2H, m), 1.37 (2H, d, J 7.8).

### Synthesis of H-GK(4-pa)G-Ahx-GK(4-pa)G-NH₂ (Core 2):

H-GK(Mtt)G-Ahx-GK(Mtt)G-resin was synthesized and 4-penytnoic acid was coupled through the Lys residues as described previously. MALDI: Calculated [M+H]+=775.9g/mol, found [M+H]+=775.6 g/mol. δ ¹H NMR (500 MHz, D2O) 4.32-4.22 (2H, m), 3.90 (2H, s), 3.87 (2H, d, J 8.5), 3.84 (4H, d, J 4.7), 3.17 (6H, t, J 6.5), 2.45 (4H, d, J 6.4), 2.39 (4H, t, J 6.6), 2.32 (2H, s), 2.28 (2H, t, J 7.5), 1.75 (4H, d, J 33.8), 1.62-1.54 (2H, m), 1.49 (6H, d, J 7.0), 1.33 (6H, dd, J 7.3, 38.6).

### Synthesis of H-GK(4-pa)G-Ahx-GK(4-pa)G-Ahx-GK(4-pa)G-NH₂ (Core 3):

H-GK(Mtt)G-Ahx-GK(Mtt)G-Ahx-GK(Mtt)G-resin was synthesized and 4-penytnoic acid was coupled through the Lys residues as described previously. MALDI: Calculated [M+H]+=1211.4 g/mol, found [M+H]+=1211.1 g/mol. δ ¹H NMR (500 MHz, D₂O) 4.32-4.18 (3H, m), 3.92 (4H, d, J 18.3), 3.87 (2H, d, J 8.9), 3.83 (6H, t, J 4.6), 3.17 (6H, d, J 6.8), 3.15 (4H, d, J 7.2), 2.45 (6H, dd, J 5.2, 7.5), 2.38 (6H, t, J 6.6), 2.32 (3H, t, J 2.4), 2.28 (4H, t, J 7.4), 1.75 (6H, d, J 34.4), 1.56 (4H, dd, J 7.7, 15.2), 1.49 (10H, d, J 6.7), 1.42-1.21 (10H, m).

### Synthesis of H-CGGRGDS-NH₂:

H-CGGRGDS-NH₂ was synthesized according to the General Peptide Synthesis procedure. MALDI: Calculated [M+H]+=650.7 g/mol, found [M+H]+=650.6 g/mol. Yield: 82%.

### Synthesis of H-CGGc[CRGDSfK(Alloc)]-NH₂:

Fmoc-C(Mmt)RGDSfK(Alloc)-resin was synthesized as described previously. The monomethoxytrityl (Mmt) group was selectively deprotected on resin according to protocol. An on-resin modified Ellman's assay was used to qualitatively determine the presence of free thiol. Peptide cyclization was achieved using thiol-ene photochemistry as described elsewhere. Briefly, the resin was swollen in DMF (10 min) and the vessel purged with Ar. DMPA (1 eq. to resin) was added and irradiated with UV light (365 nm, 20 mW cm⁻²) for 3 hours. DMPA was supplemented every 10 minutes to account for initiator consumption due to photolysis. A negative Ellman's test (indicating no thiols) was used to determine when the reaction was complete. The remaining amino acids (Cys(Trt), Gly, Gly) were coupled (HATU/NMM) using the Tribute synthesizer. MALDI: Calculated [M+H]+=1112.3 g/mol, found [M+H]=1112.2 g/mol. ¹HNMR confirmed the disappearance of vinyl protons. Yield: 15%.

### General Procedure for the Synthesis of Multivalent RGD Using Thiol-Yne Photochemistry:

Cys-containing peptides (H-CGGRGDS-NH₂ or H-CGGc[CRGDSfK(Alloc)]-NH₂) were dissolved in an appropriate solvent (0.2 M). At this concentration, H-CGGRGDS-NH₂ was solubilized in H₂O while H-CGGc[CRGDSfK(Alloc)]-NH₂ was dissolved in DMF. The appropriate amount of core molecule was added to achieve a [SH]:[alkyne] ratio of 4:1 which was held constant for all reactions. Lithium phenyl-2,4,6-trimethylbenzoylphosphinate (LAP) was used as a water soluble (used with H-CGGRGDS-NH₂) Type I photoinitiator.5 2,2-dimethoxy-2-phenyl acetophenone (DMPA) was solubilzed in DMF (used with H-CGGc[CRGDSfK(Alloc)]-NH₂). Photoinitiators were added at the following molar rations [SH]:[initiator]=50:1. The reaction mixture was purged with Ar and irradiated with UV light (365 nm, 15 mW cm⁻²) for 20 minutes. Photoinitiator was supplemented at 10 min to account for photolysis. Reactions in DMF were precipitated in chilled diethyl ether and washed (2×) and then purified using RP-HPLC. Reactions in H₂O were diluted and directly purified with RP-HPLC.

### Evaluation of Multivalent RGD Peptides:

A competitive binding ELISA was used to determine the potency of the multivalent RGD peptides. GPIIb/IIIa is known to contain the integrins αIIβ and β3, which bind RGD peptide sequences. Fibrinogen is also known to bind to these integrins. The ELISA was performed to quantitate the inhibition of bound fibrinogen in the presence of RGD peptide. The ELISA was performed as described previously.4 Briefly, GPIIb/IIIa was incubated in a 96 well Maxisorp plate (Nunc) (10 μg ml⁻¹) overnight at 4° C. The plate was blocked for non-specific interactions using BSA (3.5 wt%, 3 hr). Varying peptides concentrations and fibrinogen (40 nM) was incubated in the plate for 3 hr at room temperature. Fibrinogen antibody (HRP) (1:20,000 dilution) as added (1 hr). The substrate (2,2′-Azinobis[3-ethylbenzothiazoline-6-sulfonic acid]-diammonium salt-ABTS) was added and the absorbance measured at 405 nm. Peptide concentrations are reported as total molecule molarity as opposed to normalized [RGD].

### Results

The thiol source for sequential thiol-mediated photoreactions was the natural amino acid, cysteine. FIG. 17 shows that Linear (1) and cyclic (2) RGD were synthesized on the solid phase using MBHA Rink Amide resin. Cyclic RGD was cyclized using thiol-ene photochemistry as described above. Briefly, Fmoc-C(Mmt)RGDSfK(Alloc)-resin was synthesized. The monomethoxytrityl (Mmt) was selectively deprotected on resin following an established protocol. The thiol-ene reaction was performed in the presence of photoinitiator (2,2-dimethoxy-2-phenylacetophenone: DMPA) and UV light (365 nm, 20 mW cm-2) for 3 hr. Upon reaction completion, the Fmoc group was removed and Gly, Gly, Cys(Trt) were subsequently coupled in a step-wise manner. Cyclic RGD was isolated in 15% yield (calculated based on initial resin substitution) and characterized by MALDI-TOF mass spectrometry and 1H-NMR.

Various core molecules (n=1,2, or 3 where n indicates the number of alkynes) were prepared using traditional peptide chemistry. 4-pentynoic acid was coupled to the s-amino groups of Lys amino acids to introduce alkynes orthogonal to the peptide backbone. The thiol-yne photoreaction is capable of achieving clustered peptides where the valency is equal to 2n. All thiol-yne reactions were performed using unprotected peptides in solution. The reaction solvent was chosen based on peptide solubility at the required concentrations (˜0.2M). H-CGGRGDS-NH2 or H-CGGc[CRGDSfK(Alloc)]-NH2) were dissolved in water and dimethylformamide (DMF), respectively. The appropriate photoinitiator was selected based on its solubility in the reaction solvent; LAP, lithium phenyl-2,4,6-trimethylbenzoylphosphinate, was used for water while DMPA, 2,2-dimethoxy-2-phenyl acetophenone, was used with DMF.

Table 1 shows the various multivalent RGD derivatives that were synthesized and their corresponding molecular weight. Linear RGD derivatives (compound #s 3-5) were obtained in very good yields ranging from 77-84% yield. Cyclic RGD multimers compound #s 6 and 7 were obtained in 48% and 11% yield, respectively. Compunds #8 (cyclic RGD hexamer) was not observed. We hypothesize the reason for decreased yields with increasing n relates to steric hindrances. The bulky macrocycles may prevent, or limit, the addition of 2 thiol containing peptides to 1 alkyne. During the formation of compound #7 the dominant product obtained contained 2 additions (as opposed to 4). 1H NMR shows the presence of vinyl protons (vinyl sulfide) indicating single peptide addition to each alkyne (as opposed to double peptide addition to 1 alkyne). This collective data indicates that increasing the spatial distance between alkynes or between the thiol and cyclic peptide may result in increased yield.

By way of example, the IUPAC chemical formulas for the multivalent RGD derivatives shown in FIG. 1 are: Compound #1—C22H39N11O10S; Compound #2—C45H70N14O15S2; Compound #3—C59H103N27024S2; Compound #4—C124H214N54O49S4; Compound #5—C189H325N81O74S6; Compound #6—C105H165N33034S4; Compound #7 C216H338N66069S8.

TABLE 1
CALCULATED YIELDS OF MULTIVALENT
RGD DERIVATIVES

| | | | | Calc. | [M + H] | |
|---|---|---|---|---|---|---|
| Compound # | R | n | Initiator^{b} | MW | Found | Yield(%) |
| 1^{a} | 1 | — | — | 649.7 | 650.6 | —^{a} |
| 2^{a} | 2 | — | — | 1111.3 | 1112.2 | —^{a} |
| 3 | 1 | 1 | LAP | 1638.8 | 1639.8 | 84 |
| 4 | 1 | 2 | LAP | 3373.6 | 3374.8 | 87 |
| 5 | 1 | 3 | LAP | 5108.5 | 5110.1 | 77 |
| 6 | 2 | 1 | DMPA | 2561.9 | 2563.2 | 48 |
| 7 | 2 | 2 | DMPA | 5219.9 | 5222.5 | 11 |
| 8 | 2 | 3 | DMPA | 7877.9 | — | 0 |

^{a}Compound #s 1 and 2 correspond to linear H—CGGRGDS—NH2 and cyclic H—CGGc[CRGDSfK(Alloc)]-NH2, respectively.
^{b}Type I initiators were chosen based on peptide solubility.

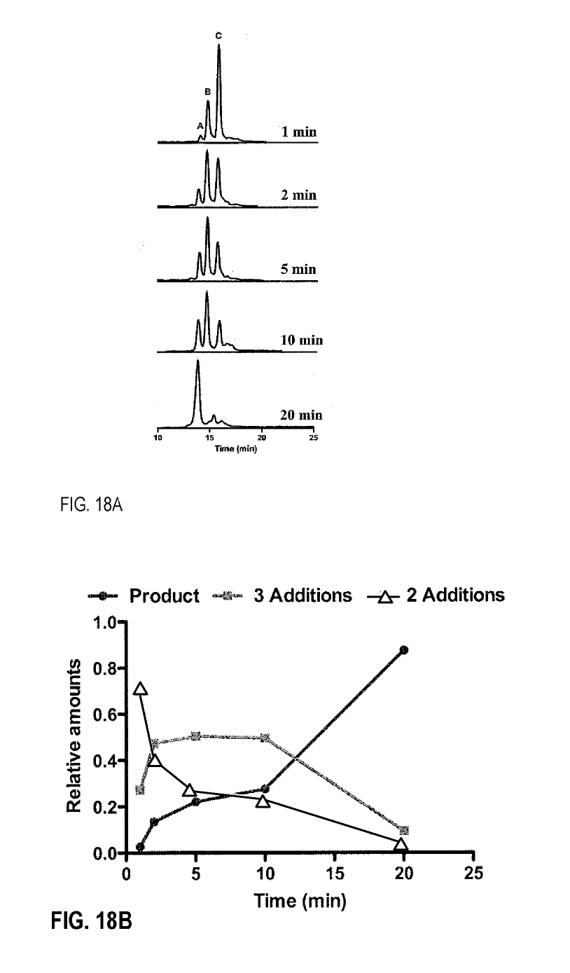

Studies were then performed to investigate the kinetics of the thiol-yne photoreaction. The formation of compound # 4 was used as a model system to explore the reaction rate. FIG. 18A shows sequential HPLC chromatograms corresponding to various time points of the reaction. The reaction is very rapid (-20 min) and the desired product (compound #4, peak A) was formed as the dominate peak. FIG. 18B shows that Peak C corresponds to a single peptide addition to a single alkyne as determined by ¹H NMR.

To evaluate the bioactivity of multivalent RGD derivatives formed via thiol-yne photochemistry, a competitive binding ELISA was performed. RGD has been shown to inhibit the binding of fibrinogen to GPIIb/IIIa. Table 2 shows the calculated IC₅₀ values for compound numbers 1-7. As expected, cyclic RGD (compound #2) formed by thiol-ene reaction exhibited enhanced potency relative to linear RGD (compound #1). Further, compound #s 7 and 5 demonstrated a decreased IC₅₀ value of 1.5-2 orders of magnitude relative to their monomeric species.

TABLE 2
IC₅₀ VALUES FOR MULTIVALENT RGD CORRESPONDING TO
INHIBITION OF FIBRINOGEN BINDING TO GPIIB/IIIA

| Compound # | R | n | IC50(μM)^{a} |
|---|---|---|---|
| 1 | 1 | — | 15.4 ± 4.2 |
| 2 | 2 | — | 0.65 ± 0.1 |
| 3 | 1 | 1 | 5.65 ± 1.5 |
| 4 | 1 | 2 | 2.42 ± 4.2 |
| 5 | 1 | 3 | 0.76 ± 0.2 |
| 6 | 2 | 1 | 0.14 ± 0.03 |
| 7 | 2 | 2 | 0.012 ± 0.003 |

^{a}Values reported as mean ± SEM. Experiments were conducted in triplicate and repeated twice.

In summary, Example 2 presents a novel strategy for the formation of multivalent peptides using thiol-yne photochemistry. The reaction is very rapid (˜20 min) and generates the desired products in relatively high yields for linear peptides (77-84%). Further, this work demonstrates that multiple thiol-mediated photoreactions (thiol-ene/thiol-yne) can be used sequentially to enhance peptide effects. This report has implications in the field of peptide chemistry and its application to peptide therapeutics.

### EXAMPLE 3

### RGD-PEG Enhanced Viability of MIN Cells

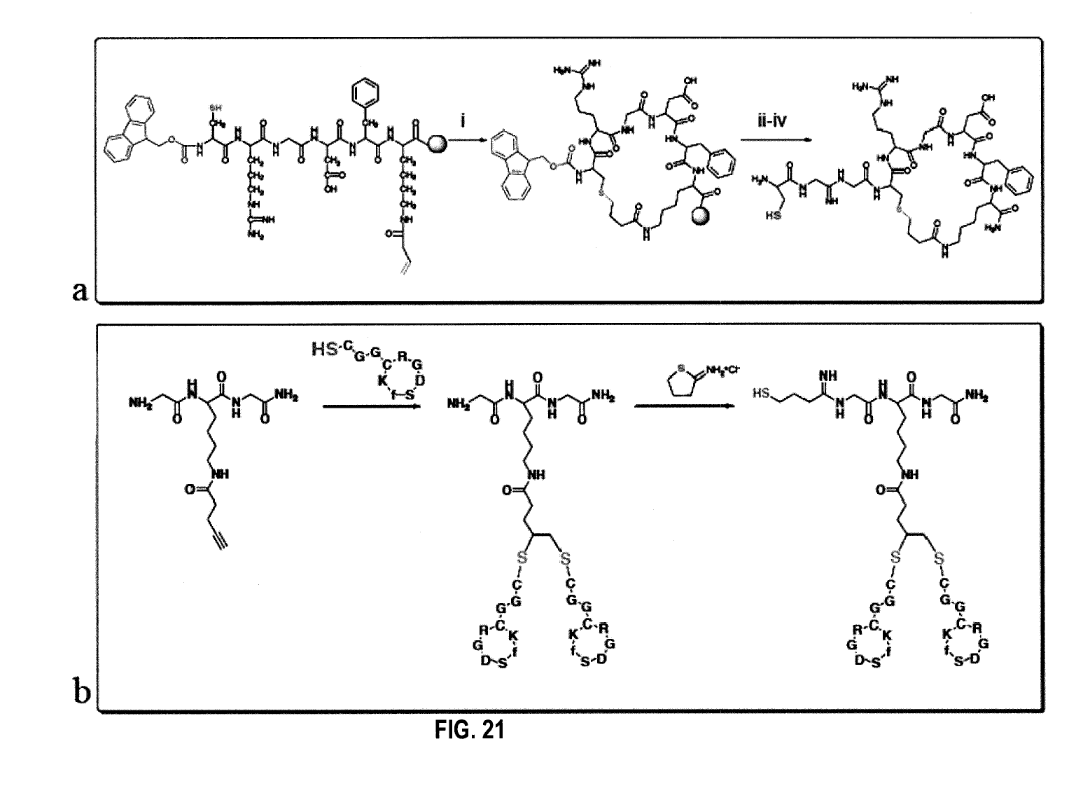

To test whether cyclic RGD, when encapsulated in PEG gels may enhance the survival of MIN6 cells, cyclic RGD was incorporated into PEG gels and applied to cell cultures of MIN6 cells. A cyclic RGD dimer was synthesized on-resin using Traut's reagent. See FIG. 21. The dimer contained a free thiol, which could be utilized for conjugation into PEG networks via thiol-acrylate photopolymerizations.

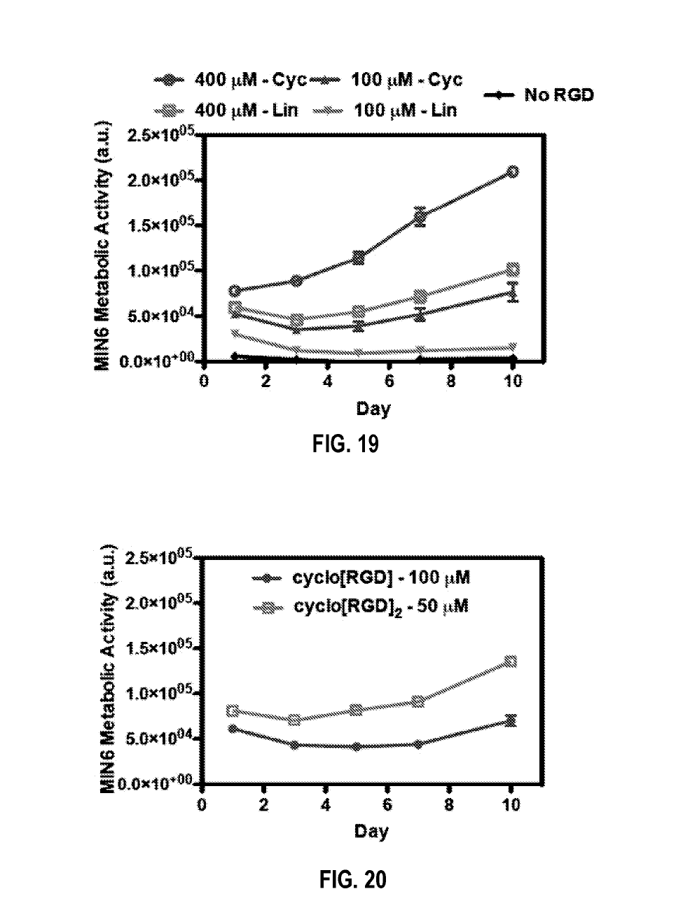

To examine whether the cyclic RGD derivatives could enhance cell viability, MIN6 cells were encapsulated at a relatively low density (5×10⁶ cells mL⁻¹, data not published) in PEG diacrylate gels with varying amounts of RGD. Cell metabolic activity was measured using an Alamar Blue assay. FIG. 19 shows initial dosing studies using either linear or cyclic RGD on MIN6 metabolic activity. Cyclic RGD (400 μM) showed significantly enhanced metabolic activity relative to its linear counterpart at the same concentration. Additionally, incorporation of 100 μM cyclic RGD resulted in increased viability, whereas 100 μM linear RGD was not able to support cell survival over the course of the 10-day study. Further, cells encapsulated in hydrogels containing the cyclic RGD dimer showed enhanced metabolic activity relative to the monomeric product (FIG. 20). Total molar RGD concentration was held constant during the study.

Photopatterning techniques may be employed to understand cell behavior when ECM mimic peptides are incorporated within spatially defined regions. Further, Weber et al. demonstrated enhanced MIN6 cell viability when encapsulated within hydrogels functionalized with matrix-derived adhesive peptides.2 In specific, laminin-derived peptide sequences (IKLLI, IKVAV, LRE, PDSGR, RGD, and YIGSR) were photopolymerized within PEG hydrogels and cell viability and insulin secretion were assayed. The results showed that cell viability was increased within peptide-functionalized hydrogels. Also, inclusion of multiple peptides provided insight to the synergistic effects. Since these sequences were all derived from a whole intact protein, laminin, constraining the conformation using macrocyclization techniques may further enhance their effect on β-cell viability.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation. Certain terms with capital or small letters, in singular or in plural forms, may be used interchangeably in this disclosure.

### REFERENCES

Some references are cited by full citation in the disclosure. Many other references are cited in the text by number only. The full citations of the references are listed below with the number corresponding to the reference numbers in the text. All references mentioned in this application, including but not limited to patent documents and scientific literature, are incorporated by reference to the same extent as though fully replicated herein.

1. L. D. Walensky, A. L. Kung, I. Escher, T. J. Malia, S. Barbuto, R. D. Wright, G. Wagner, G. L. Verdine and S. J. Korsmeyer, Science, 2004, 305, 1466.

2. G. Hummel, U. Reineke and U. Reimer, Mol Biosyst, 2006, 2, 499.

3. J. E. Oh, S. Y. Hong and K. H. Lee, J. Pept. Res., 1999, 53, 41.

4. R. Tugyi, G. Mezo, E. Fellinger, D. Andreu and F. Hudecz, J. Pept. Sci., 2005, 11, 642.

5. M. M. Madden, C. I. R. Vera, W. J. Song and Q. Lin, Chemical Communications, 2009, 5588.

6. C. E. Schafmeister, J. Po and G. L. Verdine, J. Am. Chem. Soc., 2000, 122, 5891.

7. M. Mammen, S. K. Choi and G. M. Whitesides, Angewandte Chemie-International Edition, 1998, 37, 2755.

8. J. P. Tam, Proc Natl Acad Sci U S A, 1988, 85, 5409.

9. H. C. Kolb, M. G. Finn and K. B. Sharpless, Angewandte Chemie-International Edition, 2001, 40, 2004.

10. (a) D. T. Rijkers, G. W. van Esse, R. Merkx, A. J. Brouwer, H. J. Jacobs, R. J. Pieters and R. M. Liskamp, Chem Commun (Camb), 2005, 4581; (b) I. Dijkgraaf, A. Y. Rijnders, A. Soede, A. C. Dechesne, G. W. van Esse, A. J. Brouwer, F. H. Corstens, O. C. Boerman, D. T. Rijkers and R. M. Liskamp, Org Biomol Chem, 2007, 5, 935; (c) C. B. Yim, O. C. Boerman, M. de Visser, M. de Jong, A. C. Dechesne, D. T. S. Rijkers and R. M. J. Liskamp, Bioconjugate Chem., 2009, 20, 1323.

11. (a) R. Hoogenboom, Angew Chem Int Ed Engl, 2010, DOI: 10.1002/anie.201000401; (b) A. B. Lowe, Hoyle, C. E., Bowman, C. N., J. Mater. Chem, 2010, DOI: 10.1039/b917102a.

12. (a) J. W. Chan, C. E. Hoyle and A. B. Lowe, J Am Chem Soc, 2009, 131, 5751; (b) B. D. Fairbanks, T. F. Scott, C. J. Kloxin, K. S. Anseth and C. N. Bowman, Macromolecules, 2009, 42, 211; (c) R. M. Hensarling, V. A. Doughty, J. W. Chan and D. L. Patton, J Am Chem Soc, 2009, 131, 14673; (d) D. Konkolewicz, A. Gray-Weale and S. Perrier, J Am Chem Soc, 2009, 131, 18075.

13. G. Chen, J. Kumar, A. Gregory and M. H. Stenzel, Chem Commun (Camb), 2009, 6291.

14. A. A. Aimetti, R. K. Shoemaker, C. C. Lin and K. S. Anseth, Chem Commun (Camb), 2010, DOI: 10.1039/c001375g.

15. T. Majima, W. Schnabel and W. Weber, Makromolekulare Chemie-Macromolecular Chemistry and Physics, 1991, 192, 2307.

16. P. L. Barker, S. Bullens, S. Bunting, D. J. Burdick, K. S. Chan, T. Deisher, C. Eigenbrot, T. R. Gadek, R. Gantzos, M. T. Lipari, C. D. Muir, M. A. Napier, R. M. Pitti, A. Padua, C. Quan, M. Stanley, M. Struble, J. Y. K. Tom and J. P. Burnier, J. Med. Chem., 1992, 35, 2040.

17. Barlos, K.; Gatos, D.; Hatzi, O.; Koch, N.; Koutsogianni, S. Int. J. Pept. Protein Res. 1996, 47, 148-153.

18. Badyal, J. P.; Cameron, A. M.; Cameron, N. R.; Coe, D. M.; Cox, R.; Davis, B. G.; Oates, L. J.; Oye, G.; Steel, P. G. Tetrahedron Lett. 2001, 42, 8531-8533.

19. Bycroft, B. W.; Chan, W. C.; Chhabra, S. R.; Hone, N. D. Journal of the Chemical Society-Chemical Communications 1993, 778-779.

20. Harris, R. K.; Becker, E. D.; De Menezes, S. M. C.; Granger, P.; Hoffman, R. E.; Zilm, K. W. Pure Appl. Chem. 2008, 80, 59-84.

21. D. Li and D. L. Elbert, J. Pept. Res., 2002, 60, 300.

22. K. Barlos, D. Gatos, O. Hatzi, N. Koch and S. Koutsogianni, Int. J. Pept. Protein Res., 1996, 47, 148.

23. T. Majima, W. Schnabel and W. Weber, Makromolekulare Chemie-Macromolecular Chemistry and Physics, 1991, 192, 2307.

24. Moellering, R. E.; Cornejo, M.; Davis, T. N.; Del Bianco, C.; Aster, J. C.; Blacklow, S. C.; Kung, A. L.; Gilliland, D. G.; Verdine, G. L.; Bradner, J. E. Nature 2009, 462, 182-8.

25. Besser, D.; Muller, B.; Kleinwachter, P.; Greiner, G.; Seyfarth, L.; Steinmetzer, T.; Arad, O.; Reissmann, S. Pract. Appl. Appl. Chem. 2000, 342, 537-545.

26. (a) Gilon, C.; Halle, D.; Chorev, M.; Selinger, Z.; Byk, G. Biopolymers 1991, 31, 745-750. (b) Gudmundsson, O. S.; Vander Velde, D. G.; Jois, S. D. S.; Bak, A.; Siahaan, T. J.; Borchardt, R. T. J. Pept. Res. 1999, 53, 403-413.

27. Ranganathan, D.; Haridas, V.; Kurur, S.; Nagaraj, R.; Bikshapathy, E.; Kunwar, A. C.; Sarma, A. V. S.; Vairamani, M. J. Org. Chem. 2000, 65, 365-374.

28. Shao, Y.; Lu, W. Y.; Kent, S. B. H. Tetrahedron Lett. 1998, 39, 3911-3914.

29. Meutermans, W. D. F.; Golding, S. W.; Bourne, G. T.; Miranda, L. P.; Dooley, M. J.; Alewood, P. F.; Smythe, M. L. J. Am. Chem. Soc. 1999, 121, 9790-9796.

30. Miller, S. J.; Grubbs, R. H. J. Am. Chem. Soc. 1995, 117, 5855-5856.

31. Hiroshige, M.; Hauske, J. R.; Zhou, P. J. Am. Chem. Soc. 1995, 117, 11590-11591.

32. (a) Punna, S.; Kuzelka, J.; Wang, Q.; Finn, M. G. Angewandte Chemie-International Edition 2005, 44, 2215-2220.

33. Killops, K. L.; Campos, L. M.; Hawker, C. J. J. Am. Chem. Soc. 2008, 130, 5062-+.

34. Fairbanks, B. D. S., M. P.; Halevi, A. E.; Nuttelman, C. R.; Bowman, C. N.; Anseth, K. S. Advanced Materials, In Press, doi:10.1002/adma.200901808.

35. Lee, T. Y.; Roper, T. M.; Jonsson, E. S.; Guymon, C. A.; Hoyle, C. E. Macromolecules 2004, 37, 3606-3613.

36. Fiore, M.; Marra, A.; Dondoni, A. J. Org. Chem. 2009, 74, 4422-4425.

37. (a) DeForest, C. A.; Polizzotti, B. D.; Anseth, K. S. Nat. Mater. 2009, 8, 659-664. (b) Polizzotti, B. D.; Fairbanks, B. D.; Anseth, K. S. Biomacromolecules 2008, 9, 1084-1087.

38. Haubner, R.; Gratias, R.; Diefenbach, B.; Goodman, S. L.; Jonczyk, A.; Kessler, H. J. Am. Chem. Soc. 1996, 118, 7461-7472.

39. “Protective Groups in Organic Synthesis,” Theodora W. Greene and Peter G. Wuts, editors, John Wiley & Sons, New York, 3^{rd} edition, 1999 [ISBN 0471160199]

40. Theodoridis, G. Tetrahedron 2000, 56, 2339-2358

What is claimed is:

1. A method of forming a cyclic peptide, said method comprising the steps of:
(a) providing a linear peptide having at least one free thiol group and at least one unsaturated carbon-carbon bond; and
(b) forming the cyclic peptide in a radical-mediated reaction wherein said at least one free thiol group reacts with said at least one unsaturated carbon-carbon bond.

2. The method of claim 1, wherein said linear peptide is attached to a solid support.

3. The method of claim 1, wherein said at least one unsaturated carbon-carbon bond comprises at least one carbon-carbon double bond.

4. The method of claim 1, wherein said radical-mediated reaction is a photoreaction.

5. The method of claim 1, further comprising the step of treating said linear peptide with a photoinitiator.

6. The method of claim 2, further comprising the step of cleaving the cyclized peptide from the solid support.

7. The method of claim 1, wherein said linear peptide comprises one or more cysteine residue.

8. The method of claim 1, wherein said linear peptide has a formula of:
(Aaa)ₓ-Aaa(SH)-(Aaa)_{y}-Aaa(R)-(Aaa)_{z} or (Aaa)ₓ-Aaa(R)-(Aaa)_{y}-Aaa(SH)-(Aaa)_{z},
wherein R is a side chain comprising an unsaturated carbon-carbon bond, and Aaa(SH)is an amino acid residue having at least one free thiol group, x and z each being an integer between 0 and 100, y being an integer between 2 and 100.

9. The method of claim 8 wherein R comprises a linear alkene or a cyclic alkene.

10. The method of claim 8 wherein R comprises a linear alkyne or a cyclic alkyne.

11. The method of claim 8 wherein R comprises an alkene selected from the group consisting of norbornene and CₙH₂ₙ, n being an integer between 2 and 20.

12. The method of claim 8, wherein said linear peptide has more than one Aaa(SH) residues and at least one of said Aaa(SH) residues is protected by a protective group.

13. The method of claim 12, wherein said protective group is a monomethoxytrityl (Mmt) group.

14. The method of claim 1 wherein the linear peptide is Ac-C(Mmt)RGDSfK(alkene).

15. The method of claim 1 wherein the linear peptide is Ac-C(Mmt)RGDSfK(alkyne).

16. The method of claim 1 wherein the radical-mediated reaction is selected from the group consisting of photoinitiation, thermal initiation and redox initiation.

17. A method of forming a multivalent cyclic peptide, said method comprising the steps of:
(a) coupling one or more molecules comprising an unsaturated carbon-carbon bond to one or more peptide cores; and
(b) forming the multivalent cyclic peptide by coupling two or more peptides to the peptide cores, wherein the two or more peptides have at least one free thiol group and the coupling occurs by the free thiol group attacking the unsaturated carbon-carbon bond, said two or more peptides being selected from the group consisting of a linear peptide, a cyclic peptide and combination thereof.

18. The method of claim 17, wherein step (b) occurs in the presence of a photoinitiator.

19. The method of claim 17, wherein the multivalent cyclic peptide is formed on a solid support.

20. The method of claim 17, wherein the two or more peptides comprise a peptide sequence of Arg-Gly-Asp (RGD).

21. A composition comprising a cyclic peptide, said cyclic peptide being formed through an internal thiol-ene reaction with a linear peptide having the formula of
(Aaa)ₓ-Aaa(SH)-(Aaa)_{y}-Aaa(R)-(Aaa)_{z} or (Aaa)ₓ-Aaa(R)-(Aaa)_{y}-Aaa(SH)-(Aaa)_{z},
wherein R is a side chain comprising an unsaturated carbon-carbon bond, Aaa(SH)is an amino acid residue having at least one free thiol group, x and z are each an integer between 0 and 100, y is an integer between 2 and 100, and the thiol-ene reaction taking place between said thiol group and the alkene of the R group.

22. A composition comprising a multivalent cyclic peptide, said multivalent cyclic peptide being formed by coupling two or more peptides to one or more peptide cores, wherein the two or more peptides have at least one free thiol group and the one or more peptide cores have at least one alkyne, said coupling occurring through a thiol-yne reaction between said free thiol group with the at least one alkyne, said two or more peptides being formed through an internal thiol-ene reaction with a linear peptide having the formula of:
(Aaa)ₓ-Aaa(SH)-(Aaa)_{y}-Aaa(R)-(Aaa)_{z} or (Aaa)ₓ-Aaa(R)-(Aaa)_{y}-Aaa(SH)-(Aaa)_{z},
wherein R is a side chain comprising an unsaturated carbon-carbon bond, Aaa(SH)is an amino acid residue having at least one free thiol group, x and z are each an integer between 0 and 100, y is an integer between 2 and 100, and the thiol-ene reaction taking place between said thiol group and the alkene of the R group.

23. A kit for forming a cyclic peptide, said kit comprising a linear peptide, a solid suuport, and a photoinitiator,
wherein said linear peptide is attached to said solid support, and said linear peptide having at least one free thiol group and at least one unsaturated carbon-carbon bond.

24. The kit of claim 23, wherein said linear peptide comprise a peptide sequence of Arg-Gly-Asp (RGD).
